(19) United States
(12) Patent Application Publication
(10) Pub. No.: US 2005/0059696 A1
(43) Pub. Date: Mar. 17, 2005

(54) Process for the recovery of S -(+)-methyl- (2-chlorophenyl)- (6,7-dihydro- 4H-thieno [3,2-c] pyrid-5-yl) acetate hydrogen sulfate (clopidogrel bisulfate) from its (R) and mixture of (R) and (S)- isomers

(75) Inventors: Manne Satyanarayana Reddy, Hyderabad (IN); Sajja Eswaraiah, Hyderabad (IN); Anumula Raghupathi Reddy, Hyderabad (IN); Alla Sampath, Karimnagar (IN)

Correspondence Address: LADAS & PARRY, 26 WEST 61ST STREET, NEW YORK, NY, 10023, US

(73) Assignees: DR. REDDY'S LABORATORIES LIMITED; DR. REDDY'S LABORATORIES, INC.

(21) Appl. No.: 10/842,702

(22) Filed: May 10, 2004

(30) Foreign Application Priority Data
May 8, 2003 (IN) 389/MAS/2003

(51) Int. Cl. C07D 498/02; A61K 31/4743

(52) U.S. Cl. 514/301; 546/114

(57) ABSTRACT

A process for the recovery of compound of formula (I) 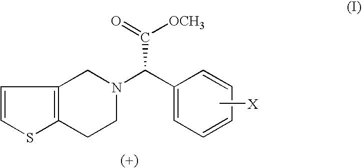 where X represents hydrogen, fluoro, chloro, bromo or iodo atom, preferably 2-chloro which comprising the steps of f. preparing compound (−) or (±)-(2-chloro phenyl)-(6,7-dihydro-4H-thieno[3,2-c]pyrid-5-yl)acetate methyl ester hydrogen sulfate from its corresponding camphorsulfonic acid salt compound. g. transforming the obtained compound of step (a), into the compound of (2-chlorophenyl)-(6,7-dihydro-4H-thieno[3,2-c]pyrid-5-yl)acetic acid. h. converting the compound of step (b) into racemic compound (±)-(2-chloro phenyl)-(6,7-dihydro-4H-thieno[3,2-c]pyrid-5-yl)acetate methyl ester hydrogen sulfate. i. resolving the obtained racemic compound of step (c), into the optically active (+)-(2-chloro phenyl)-(6,7-dihydro-4H-thieno[3,2-c]pyrid-5-yl)acetate methyl ester camphor sulfonic acid salt. j. further transforming the optically active (+) form compound of step (d) into their pharmaceutically acceptable salts.

### FIELD OF THE INVENTION

The present invention relates to a process for the recovery of thieno[3,2-c]pyridine derivatives of general formula (I) and salts thereof in optically active S-(+) form, here in after called S-(+) form as (+) form, from their stereoisomeric mother liquors of R-(−) form, here in after called R-(−) form as (−) form, or variable mixture of S-(+) and R-(−) form of general formula (II) and its salts thereof, preferably the salts may be camphorsulfonic acid salt. The compounds of formula (I) and (II) where X represents either a hydrogen or halogen atom 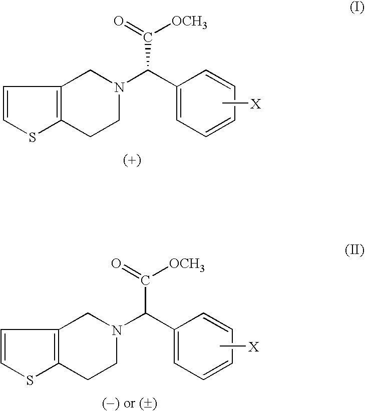
such as fluorine, chlorine, bromine or iodine, preferably X represents 2-chloro.

### BACKGROUND OF THE INVENTION

Thieno[3,2-c]pyridine derivatives disclosed in FR 2,215,948, FR 2,530,247 and FR 2,612,929 are pharmacologically active and have significant anti-aggregating and anti-thrombotic properties, one such example is Clopidogrel. Later it was found that the biological activity resides only with (S)-(+)-stereo isomer (U.S. Pat. No. 4,847,265) and its pharmaceutically acceptable salts.

The recovery process of S-(+)-Clopidogrel bisulfate from its unwanted stereoisomers R-(−)-Clopidogrel or variable mixture of (+) and (−)Clopidogrel was not mentioned in the above said patents. Later in WO 02/059128 A2 patent the process of recycle of unwanted stereo isomer of Clopidogrel and its intermediates by racemisation was mentioned in detailed description of invention, but in the experimental section the racemisation process was given for intermediates only. In the present invention the recovery process mentioned more particularly for S-(+)-Clopidogrel from its unwanted stereoisomers. By this recovery process, the overall production cost of S-(+)Clopidogrel will reduce and as well as environmental point of view it reduces the effluent load.

Consequently the present invention aims to provide an inexpensive and commercially viable process to prepare compounds of formula (I) in good yields.

### SUMMARY OF THE INVENTION

The present invention relates to a convenient process for the recovery of (S)(+)-Methyl-(2-chlorophenyl)-(6,7-dihydro-4H-thieno[3,2,-c]pyrid-5-yl)acetate hydrogen sulfate[(S)(+)Clopidogrel bisulfate] of formula (I).

The process of the present invention comprises the conversion of (−)camphor sulfonic acid salt of formula (II) into free base followed by salt formation in the presence of sodium carbonate and sulfuric acid resulted Methyl(±)-(2-chlorophenyl)-(6,7-dihydro-4H-thieno[3,2-c]pyrid-5-yl)acetate hydrogen sulfate. Which on hydrolysis with sodium hydroxide resulted the (2-chlorophenyl)-(6,7-dihydro-4H-thieno[3,2-c]pyrid-5-yl)acetic acid. The obtained compound on esterification with dimethyl sulfate in the presence of sodium hydroxide, followed by salt formation in the presence of sulfuric acid resulted the (±)-Methyl-(2-chlorophenyl)-(6,7-dihydro-4H-thieno[3,2-c]pyrid-5-yl)acetate hydrogen sulfate. This compound on resolution with camphor sulfonic acid afforded the (+)-Methyl-(2-chlorophenyl)-(6,7-dihydro-4H-thieno[3,2-c]pyrid-5-yl)acetate camphor sulfonic acid salt, which on basification with sodium carbonate resulted the corresponding free base, this free base on reaction with sulfuric acid in secondary butanol resulted (+)-Methyl-(2-chlorophenyl)-(6,7-dihydro-4H-thieno[3,2-c]pyrid-5-yl)acetate hydrogen sulfate[(S)(+)-Clopidogrel bisulfate]. This obtained compound exhibits as crystalline form-I.

The process of the present invention is alternative, cost effective, operational friendly and Eco-friendly.

### DETAILED DESCRIPTION OF THE INVENTION

Accordingly the present invention provides a process for recovery of the compounds of formula (I), in optically active (+) form and their salts from their stereoisomers. The compounds of formula (I) where X represents either hydrogen or a halogen atom such as fluorine, chlorine, bromine or iodine. More particularly, the present invention provides a process to recover Clopidogrel bisulfate from its (−) form or variable mixture of (+) and (−) form and their slats. Most particularly, the present invention provides a process to recover Clopidogrel bisulfate in its crystalline form I and II, from its (−) form or variable mixture of (+) and (−) form and their slats thereof.

The process to prepare compounds of formula (I) or its salts, uses compounds of formula (II) or its salts in either (−) form or a variable mixture of (+) and (−) forms, as outlined in Scheme-1.

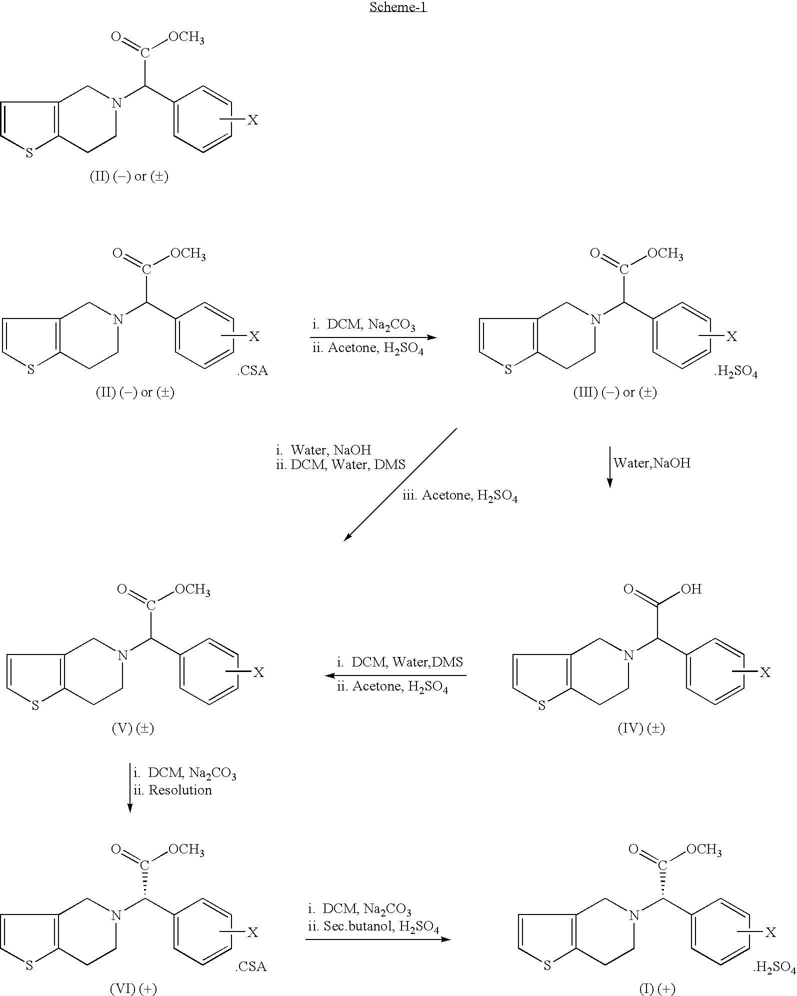

1. The present invention provides a process for the recovery of compounds of formula (I) and their salts as shown in scheme-1, which comprises:

2. preparing compound of formula (III), (−) or (±)-(2-chloro phenyl)-(6,7-dihydro-4H-thieno[3,2-c]pyrid-5-yl)acetate methyl ester hydrogen sulfate from compound of formula (II).

3. transforming the compound of formula (III) in either variable mixture of (+) and (−) forms or optically active (−) form or its salts, into the compound of formula (IV) (2-chlorophenyl)-(6,7-dihydro-4H-thieno[3,2-c]pyrid-5-yl)acetic acid.

4. converting the compound of formula (IV) into racemic compound of formula (V), (±)-(2-chloro phenyl)-(6,7-dihydro-4H-thieno[3,2-c]pyrid-5-yl)acetate methyl ester hydrogen sulfate.

5. resolving the racemic compound of formula (V) into the compound of formula (VI), in optically active (+) form.

6. further transforming the optically active (+) form compound of formula (VI) into their pharmaceutically acceptable salts.

Alternatively the compound of formula (III) in either racemic or optically active (−) form can be directly transformed into corresponding compound of formula (V) in racemic without isolation of compound of formula (IV).

Optionally suitable acid addition salts of the intermediates of formula II, III, V and VI may be used in the above-mentioned process. Suitable acids used may be selected from acetic, benzoic, fumaric, maleic, citric, tartaric, methanesulfonic, ethanesulfonic, benzenesulfonic, p-toluene sulfonic, camphorsulfonic, mandelic, hydrochloric, hydrobromic, sulfuric acids and the like

The process to prepare compounds of formula (III) comprises basification of diastereomeric salt (formula II) by using suitable base reagent, such as Na₂CO₃, NaOH and KOH in the presence of a mixture of water and suitable organic solvent such as dichloromethane, chloroform, toluene, and ethylacetate under stirring at the temperature range 0° C. to 50° C. and further conversion to sulfuric acid salt.

Preparation of compound of formula (IV) involves the hydrolysis of compound of formula (III) with mineral acids such as HCl, HBr, and sulfuric acid or with bases, such as NaOH, KOH, NaH, K⁺-t-BuO⁻, KH and the like in suitable solvent medium such as water, acetonitrile, and (C₁-C₄) alcohol at the temperature ranges from 20° C. to 100° C., preferably from 70° C. to 100° C.

The reagents used in the above process may range from equimolar to 5 moles. The duration of reaction may range from 5-10 hours.

The process to convert the compound of formula (IV) into methyl ester of formula (V) may be carried out in the presence of reagents, which include acid or a base. The preparation of methyl ester in acidic conditions involves reaction with methanol in presence of acids, such as sulfuric acid, SOCl₂, POCl₃ and the like. The preparation of methyl ester in basic conditions involves the reaction with DMS in presence of bases, such as KOH, NaOH, K⁺-t-Buo⁻, NaH, KH, tri ethyl amine, trimethyl amine and the like, in suitable solvent, like DMF, methanol, toluene, DCM, chloroform or mixture of water and DCM. With or without a phase transfer catalyst the reaction is carried out in biphasic solvent system. Suitable phase transfer catalyst used in such a case may be tetra butyl ammonium halide, benzyl tri methyl ammonium halide, and the like, at the temperature range from 25° C. to 100° C., preferably from 25° C. to 35° C.

Compound of formula (VI) obtained by resolution of compound of formula (V). The process of resolution comprises of dissolving the racemic mixture in suitable solvent and addition of a suitable chiral reagent. Suitable solvent is selected on the basis whether the diastereomeric salt precipitates out differently. The separation may result simply by stirring at a suitable temperature in a solvent until one of the salts preferentially precipitate out. Purification of diastereomeric salt is possible by refluxing in a suitable solvent. Preferably in aqueous acetone, the free base is liberated from its salt using a suitable base reagent. The diastereomeric salt is dissolved or suspended in a mixture of water and organic solvent and is neutralized with a base under stirring, the free base is obtained after separation of aqueous layer and evaporation of the organic solvent.

Suitable base reagent for the hydrolysis of diastereomeric salt includes sodium carbonate, potassium carbonate, sodium hydrogen carbonate and potassium hydrogen carbonate in aqueous medium at temperature varying between 5° C. to 25° C.

The solvents used during the resolution can include solvents or mixture there of such as (C₁-C₄) alcohol, (C1-C4) ketone, ethyl acetate, methyl acetate, methyl ethyl ketone, DMF, actonitrile, propionitrile, THF, dioxane and the like. The solvent used optionally may contain water up to 2%, but presence of water or its amount is not critical. Suitable temperature range for the resolution includes temperature from 0° C. to reflux temperature of the solvent used, the acid chiral reagents used for the resolution include tartaric acid, mandelic acid, lactic acid, camphorsulfonic acid, maleic acid, amino acids and the like.

Finally the desired or pharmaceutically acceptable salt of compound of formula (I) can be formed from the corresponding stereoisomer and a suitable acid. The optically pure(s)-(+) compound of formula (I), is converted into its bisulfate salt using sulfuric acid in an appropriate solvent at suitable temperature to afford (+)-Clopidogrel bisulfate. The obtained (+)-Clopidogrel bisulfate by the above process shows crystalline form-I.

In the above process suitable acid may be hydrochloric, hydrobromic, sulfuric acid in suitable solvent such as C₁-C₄ ketone, C₁-C₄ alcohol, ethyl acetate, dichloromethane, acetonitrile, methyl acetate. The temperature ranges from 0° C. to reflux temperature of the solvent used. The reagents used in the process may range from 0.9 mole to 1.2 mole ratio.

This manufacturing process to recycle the compounds of general formula (I) as shown in Scheme 1, has following advantages:

Simple and readily available reagents/chemicals are used

Milder reaction conditions are employed in various steps.

It is possible to recemise the unwanted isomer, there by enhances the efficiency and reduces the effluent load.

It is continual process

The above factors contribute to improve the cost effectiveness of the process described.

The process described in the present invention is demonstrated in the examples illustrated below. These examples are provided as illustration only and therefore should not be construed as limitation to the scope of the invention.

### EXAMPLE: 1

### Preparation of (−) or variable mixture of (+) and (−)Methyl(2-chlorophenyl)-(6,7-dihydro4H-thieno[3,2-c]pyrid-5yl)acetate hydrogen sulfate (III)

Take mother liquor of (−) or variable mixture of (+) and (−) Methyl (2-chlorophenyl)-(6,7-dihydro-4H-thieno[3,2-c]pyrid-5-yl)acetate camphor sulfonate (equivalent to 182 g) and distill off completely at below 60° C. Cool to 30-35° C., add 100 ml of dichloromethane and 100 ml of water. Adjust the pH to 7.5-8.0 with 10% Na₂CO₃ solution (200 ml). The lower organic layer was separated and aqueous layer was extracted with dichloromethane (2×100 ml). Washed the organic layer with water (2×50 ml). Concentrated the organic layer under reduced pressure and dissolved in 300 ml of acetone, cooled to 20-25° C. Slowly added 17 ml of sulfuric acid and stirred for 1 hr at 20-25° C. Filtered the solid product and washed with 50 ml of acetone. The amount of solid product obtained was 110 g (80%).

### EXAMPLE: 2

### Preparation of (±)-(2-chlorophenyl)-(6,7-dihydro-4H-thieno[3,2-c]pyrid-5-yl)acetic acid (IV)

Charged 5 g (5.2 mol) of NaOH flakes into 30 ml of water and stirred for clear solution. To this clear solution charged 10 g (1.0 mol) of (−) or variable mixture of (+) and (−) Methyl-(2-chlorophenyl)-(6,7-dihydro-4H-thieno[3,2-c]pyrid-5-yl)acetate hydrogen sulfate (III). Which was heated to reflux for 5 hr. The reaction mixture was cooled to 30-35° C., charge 10 ml of water. Acidified the reaction mass with 7 ml (2.819 mol) of hydrochloric acid. The resulted reaction mixture was stirred for 1 hr. Filtered the solid product and washed with 2.5 ml of water. The yield of title product was 7.0 g (95.6%)

SOR: −0.418° (C=1.68,NaOH solution)

### EXAMPLE: 3

### Preparation of (±)Methyl-(2-chlorophenyl)-(6,7-dihydro-4H-thieno[3,2-c]pyrid-5-yl)acetate hydrogen sulfate (V)

Charged 200 ml of DCM and 20 g (1.0 mol) of (±)-(2-chlorophenyl)-(6,7-dihydro-4H-thieno[3,2-c]pyrid-5-yl)acetic acid (IV). Slowly added 100 ml of 2% NaOH solution, the resulted reaction mixture was cooled to 0-5° C. and charged 1.2 g of tetra butyl ammonium bromide, then slowly added 8 ml (1.30 mol) of dimethyl sulfate. Temperature raised to 25-30° C., which was stirred for 12 hr. Charged 120 ml of water and bottom organic layer was separated. Aqueous layer was extracted with dichloromethane (2×100 ml), concentrated the organic layer. To this residue charged 85 ml of acetone and cooled to 20-25° C. To the resulted solution added 3.6 ml (1.04 mol) of sulfuric acid and stirred for 1 hr. Filtered the compound and washed with 5 ml of acetone. Charged the wet compound into a solution of 95 ml of acetone and 5 ml of water. Heated the reaction mass to reflux for 30-45 min. Cooled to 10-15° C. and stirred for 30-45 min. Filtered the compound and washed with 5 ml of acetone. The yield of title product is 17.6 g (64.0%).

The product obtained was characterised by using IR spectrum, mass and ¹H-NMR, which are as given below.

IR spectrum (cm⁻¹): 1754 (C═O)

Mass spectrum (m/z): 322(M+1)

¹H-NMR(CDCl₃): δ7.5-7.8(4H,m), δ1.3(3H,S), δ3.8(2H,br,s), δ2.8(2H,br,s)

### EXAMPLE: 4

### Preparation of (±)-Methyl-(2-chlorophenyl)-(6,7-dihydro-4H-thieno[3,2-c]pyrid-5-yl)acetate hydrogen sulfate (V)

To a solution of 500 ml of water and 50 g (5.2 mol) of sodium hydroxide was added 100 g (1.0 mol) of (−) or variable mixture of (+) and (−)Methyl-(2-chlorophenyl)-(6,7-dihydro-4H-thieno[3,2-c]pyrid-5-yl)acetate hydrogen sulfate (III), which was heated to 80-85° C. and stirred for 5-6 hr. Cooled to 20-25° C., Slowly charged 70 ml (2.819 mol) of hydrochloric acid to adjust the pH, the resulted reaction mixture was stirred for 1-1 1/2 hr at 20-25° C. Filtered the solid compound and washed with 50 ml of water, suck dried for 30 min. Charged this wet compound into 325 ml of dichloromethane and cooled to 0-5° C. A solution of 9.75 g. (1.02 mol) of sodium hydroxide and 300 ml of water was added to the reaction mass. Charged 4.0 g of tetra butyl ammonium bromide, then slowly charged 26 ml (1.154 mol) of dimethyl sulfate to reaction mixture at 0-5° C. Raised the temperature to 25-30° C. and stirred for 10-12 hr. Charged 400 ml of water and separated the bottom organic layer, extracted the aqueous layer with dichloromethane (2×150 ml). Washed the organic layer with water (2×50 ml) and concentrated under reduced pressure. The resulted residue was dissolved in 300 ml of acetone and cooled to 20-25°. Slowly charged 11.5 ml (0.9 mol) of sulfuric acid and stirred for 1-1½ hr. Filtered the product and washed with 15 ml of acetone, suck dried for 10-15 min. This solid product was charged into mixture of 300 ml of acetone and 16 ml of water, which was heated to reflux for 30-45 min. Cooled to 10-15° C. and stirred for 30-45 min. Filtered the product and washed with 30 ml of acetone. Dried the product at 60° C. The yield of title product is 61 g (61%).

### EXAMPLE: 5

### Preparation of (+)-Methyl-(2-Chlorophenyl)-(6,7-dihydro-4H-thieno[3,2-c]pyrid-5-yl)acetate camphor sulfonic acid salt (VI)

Charged 200 g (1.0 mol) of (±)-Methyl-(2-chloro phenyl)-(6,7-dihydro-4H-thieno[3, 2-c]pyrid-5-yl)acetate hydrogen sulfate into 800 ml of dichloromethane and cooled to 0-5° C. Reaction mass pH was adjusted to 7.5-8.0 with a solution of 62.5 g (1.23 mol) of sodium carbonate and 514 ml of water and stirred for 30 min. Separated the bottom organic layer and extracted the aqueous layer with dichloromethane (2×200 ml). Washed the organic layer with water 2×175 ml) and concentrated under reduced pressure at below 60° C., then cooled to 30-35° C. Charged 800 ml of acetone to the residue and stirred for clear solution.

To this solution added 7 ml of water and 115 g (1.04 mol) of camphor sulfonic acid. Stirred for 30-45 min and charged 0.75 g of (+)-Methyl-(2-Chlorophenyl)-(6,7-dihydro-4H-thieno[3,2-c]pyrid-5-yl)acetate camphor sulfonic acid salt. The resulted reaction mixture was stirred for 16-18 hr at 30-35° C. Filter the solid product and washed with 75 ml of acetone, dried at 70-75° C. The yield of the title product is 81 g (61.4%).

The product obtained was characterised by using IR Spectrum, mass and ¹H-NMR, which are as given below.

Melting range: 162-164° C.

SOR: +24.09⁰ (C=1.68, methanol)

IR Spectrum (Cm⁻¹): 1755 (C═O)

Mass Spectrum (m/z): 322 (M+1)

¹H-NMR (CDCl₃): δ 7.5-8 (4H, m), δ 6.8 (1H, d), δ 7.3 (1H,s)

### EXAMPLE: 6

### Preparation of (+)-Methyl-(2-chloro phenyl)-(6,7-dihydro-4H-thieno[3,2-c]pyrid-5-yl)acetate camphor sulfonic acid salt (VI)

Charged 36 g (1.0 mol) of (−)camphor sulfonic acid to a solution of 100 ml of isopropyl alcohol and 50 g. (1.0 mol) of (±)-Methyl-(2-chloro phenyl)-6,7-dihydro-4H-thieno[3,2-c]pyrid-5-yl)acetate (free base of compound of formula V). The reaction mixture was seeded with 0.4 g. of (+)-Methyl-(2-chlorophenyl)-(6,7-dihydro-4H-thieno[3,2-c]pyrid-5-yl)acetate camphor sulfonic acid salt and stirred for 20 hr at 20-25° C. Cooled the reaction mass to 0-5° C. and stirred for 1 hr. Filtered the title product and washed with isopropyl alcohol (2×10 ml). The yield is 23.0 g

(53.48%).

SOR: +24⁰ (C=1.68, methanol)

### EXAMPLE: 7

### Preparation of (+)-Methyl-(2-chlorophenyl)-(6,7-dihydro-4H-thieno[3,2-c]pyrid-5-yl)acetate camphor sulfonic acid salt (VI)

Charged 36 g (1.0 ml) of (−)camphor sulfonic acid to a solution of 100 ml of acetone and 50 g. (1.0 mol.) of (±)-Methyl-(2-chlorophenyl)-(6,7-dihydro-4H-thieno[3,2-c]pyrid-5-yl)acetate (free base of compound of formula V). The reaction mixture was seeded with 0.4 g. of (+)-Methyl-(2-chlorophenyl)-(6,7-dihydro-4H-thieno[3,2-c]pyrid-5-yl)acetate camphor sulfonic acid salt and stirred for 20 hr at 20-25° C. Cooled the reaction mass to 0-5° C. and stirred for 1 hr. Filtered the title product and washed with acetone (2×10 ml). The yield is 26 g (60.4%).

Melting range: 158-161° C.

SOR: +24.03° (C=1.68, methanol)

### EXAMPLE: 8

### Purification of (+)-Methyl-(2-chloro phenyl)-(6,7-dihydro-4H-thieno[3,2-c]pyrid-5-yl)acetate camphor sulfonic acid salt (VI)

Charge 250 g of (+)-Methyl-(2-chloro phenyl)-(6, 7-dihydro-4H-thieno[3,2-c]pyrid-5-yl)acetate camphor sulfonic acid salt into a solution of 2500 ml of acetone and 43.75 ml of water. Heated to reflux for clear solution, and stirred for 10-15 min. Cooled the reaction mixture to 0-5° C. and stirred for 20-30 min. Filtered the solid compound and washed with 250 ml of acetone, dried at 70-75° C. The yield is 187 g (75%).

Chiral Purity: 100%

### EXAMPLE: 9

### Preparation of Polymorph I of (+)-Methyl-(2-chlorophenyl)-(6,7-dihydro-4H-thieno[3,2-c]pyrid-5-yl)acetate hydrogen sulfate (I)

20 g (1.0 mol) of (+)-Methyl-(2-chloro phenyl)-(6,7-dihydro-4H-thieno[3,2-c]pyrid-5-yl)acetate camphor sulfonic acid salt was dissolved in 86 ml of dichloro methane and cooled to 0-5° C., then basified with 20 ml of 10% sodium carbonate solution. Separated the bottom organic layer and extracted the aqueous layer with dichloro methane (2×30 ml). Washed the organic layer with water (2×30 ml) and concentrated under reduced pressure. Dissolved the resulted residue in 176 ml of sec.butanol charged 1.0 g of basic carbon. Stirred for 30-45 min at 25-35° C. and filtered the reaction mass. Washed with 50 ml of sec.butanol and charged the total filtrate into the RBF. Cooled the reaction mass to 10-15° C. Added 1.93 ml (1.0 ml) of concentrated sulfuric acid to the reaction mass at 10-15° C., then temperature raised to 20-30° C. with in 20-30 min and stirred for 13-18 hr. Filtered the solid compound and washed with 20 ml of sec. butanol. Suck dried the compound and again washed with 20 ml of cyclohexane. The yield of the product is 10.7 g (70%).

The product obtained was characterised by different physico-chemical characteristics, which are as given below.

Melting point: 184±2° C.

SOR: +55.08° (C=1.89, Methanol)

Chiral purity: 99.947%

IR and XRD were found matching to that reported in the literature (WO 99/65915).

1. A process for the recovery of compound of formula (I) 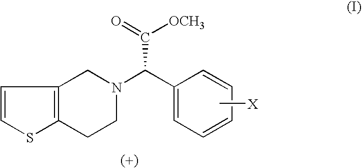
where X represents hydrogen, fluoro, chloro, bromo or iodo atom, preferably 2-chloro which comprising the steps of
a. preparing compound (−) or (±)-(2-chloro phenyl)-(6,7-dihydro-4H-thieno[3,2-c]pyrid-5-yl)acetate methyl ester hydrogen sulfate from its corresponding camphorsulfonic acid salt compound:
b. transforming the obtained compound of step (a), into the compound of (2-chlorophenyl)-(6,7-dihydro-4H-thieno[3,2-c]pyrid-5-yl)acetic acid.
c. converting the compound of step (b) into racemic compound (±)-(2-chloro phenyl)-(6,7-dihydro-4H-thieno[3,2-c]pyrid-5-yl)acetate methyl ester hydrogen sulfate.
d. resolving the obtained racemic compound of step (c), into the optically active (+)-(2-chloro phenyl)-(6,7-dihydro-4H-thieno[3,2-c]pyrid-5-yl)acetate methyl ester camphor sulfonic acid salt.
e. further transforming the optically active (+) form compound of step (d) into their pharmaceutically acceptable salts.

2. A process according to claim 1, where the starting compound, methyl-(2-chlorophenyl)-(6,7-dihydro-4H-thieno[3,2-c]pyrid-5-yl)acetate camphor sulfonic acid salt in either, optically active (−) form or variable mixture of (+) and (−) form.

3. A process according to claim 2, the compound may be the salt of tartaric acid, mandelic acid, lactic acid, amino acids, maleic acid or camphor sulfonic acid preferably camphor sulfonic acid salt.

4. A process for the preparation of compound obtained according to claim 1(a), and its salts, like acetic acid, benzoic acid, fumaric, maleic citric, tartaric, methane sulfonic, ethane sulfonic, benzene sulfonic, p-toluene sulfonic, camphor sulfonic, hydrochloric, sulfuric, hydrobromic, more particularly sulfuric acid salt.

5. A process according to claim 1(b ), the preparation of compound (2-chloro phenyl)-(6,7-dihydro-4H-[3,2-c]Pyrid-5-yl)acetic acid, in racemic form.

6. A process according to claim 1(b), using a base such as NaH, KH, NaOH, KOH preferably NaOH in a suitable solvent at a suitable temperature.

7. A process according to claim 6, where in suitable temperature is from 70° C. to 100° C.

8. A process according to claim 1(c), where in preparation of compound is in racemic form.

9. A process according to claim 1(c), where the suitable methylating agent is dimethylsulfate in suitable solvent, with a suitable phase transfers catalyst, in the presence of a suitable base at a suitable temperature.

10. A process according to claim 9, where in suitable solvent is methanol, dimethylformamide, dichloromethane, Chloroform and toluene preferably dichloromethane.

11. A process according to claim 9, where in suitable phase transfer catalyst is tertiary butyl ammonium halide, benzyl tri methyl ammonium halide, where halide represents fluoro, chloro bromo or iodo preferably tertiary butyl ammonium bromide.

12. A process according to claim 9, where in suitable base is KOH, NaOH, NaH, KH, K⁺-t-BuO⁻, triethyl or trimethyl amine preferably NaOH.

13. A process according to claim 9, where in suitable temperature is from 25° C. to 1001° C.

14. A process according to claim 1(c), where in preparation of compound is in its salt such as acetic, benzoic, fumaric, maleic, citric, tartaric, methane sulfonic, ethane sulfonic, benzene sulfonic, p-toluene sulfonic, camphor sulfonic, hydrochloric, sulfuric, hydrobromic, more preferably sulfuric acid salt.

15. A process according to claim 1(d), where in the preparation of compound is in optically active (+) form.

16. A process according to claim 1(d), where the suitable resolving agent is tartaric acid, mandelic acid, lactic acid, camphor sulfonic acid, maleic acid, amino acids, more preferably (−) camphor sulfonic acid in suitable solvent at a suitable temparature.

17. A process according to claim 16, the suitable solvent is C₁-C₄ alcohol, ethyl acetate, methyl acetate, keto solvents like acetone, propanone, methyl ethyl ketone, methyl isobutyl ketone preferably acetone, dimethyl form amide, acetonitrile, propeonitrile, THF, dioxane and halogenated hydrocarbons such as dichloromethane, dichloro ethane and chloroform preferably dichloro methane.

18. A process according to claim 16, the suitable temparature is from 0° C. to reflux temperature of the solvent used.

19. A process according to claim 16, where in said resolving agent used in 1:1 mole ratio.

20. A process of purification of compound (+)-(2-chloro phenyl)-(6,7-dihydro-4H-thieno[3,2-c]pyrid-5-yl)acetate methyl ester camphor sulfonic acid salt in aqueous acetone, and the percentage of water is 1.0% to 2.5%, more preferable is 1.75% of water.

21. A process for the preparation of compound of according to claim 1(e), in pharmacologically active (+) form and its pharmaceutically acceptable salts in suitable solvents at a suitable temparature.

22. A process according to claim 21, where in pharmaceutically acceptable salts such as, hydrochloric, hydrobromic, sulfuric, more preferably sulfuric acid salts.

23. A process according to claim 21, where in the suitable solvent is C₁-C₄ ketone, C₁-C₄ strait or branched chain alcohols, ethylacetate, methyl acetate, acetonitrile, propionitrile, halogenated hydrocarbons such as dichloromethane, dichloro ethane and chloroform preferably dichloromethane.

24. A process according to claim 21, where in the suitable temperature is from 0° C. to reflux temperature of the used solvent.

25. A process according to claim 22, where in the suitable mole ratio of the acid is from 0.9 mole to 1.2 mole.

26. The process for the recovery of S-(+)-methyl-(2-chlorophenyl)-(6,7-dihydro-4H-[3,2-c]pyrid-5-yl)acetate hydrogen sulfate according to claim 1 is in crystalline form I.

27. The process for the recovery of S-(+)-methyl-(2-chlorophenyl)-(6,7-dihydro-4H-[3,2-c]pyrid-5-yl)acetate hydrogen sulfate is substantially as here in described and exemplified.
